(19) United States
(12) Patent Application Publication
(10) Pub. No.: US 2003/0206882 A1
(43) Pub. Date: Nov. 6, 2003

(54) Fatty acid sanitizer

(76) Inventors: Francis L. Richter, Lino Lakes, MN (US); Duane Reinhardt, Maplewood, MN (US); David McSherry, Minneapolis, MN (US); Keith Lascotte, Maplewood, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/138,342

(22) Filed: May 3, 2002

(51) Int. Cl. A61K 31/19; A61K 7/075; A61K 7/08

(52) U.S. Cl. 424/70.24; 514/557

(57) ABSTRACT

An antimicrobial composition comprising at least one aliphatic antimicrobially effective C₆ to C₁₀ fatty acid and at least one coupling agent and a vicocity modifying agent. The composition finds utility for use in teat dips and skin sanitizing and or cleaning.

### FIELD OF THE INVENTION

The present invention relates to an antimicrobial composition having at least one aliphatic short chain antimicrobially effective C₆-C₁₀ fatty acid and a coupler. A viscosity modifying agent and other additives may optionally be employed.

### BACKGROUND OF THE INVENTION

Antimicrobial compositions are used for the prevention and control of various microorganisms including bacteria, fungi, protozoa, and so forth. Topical antimicrobial compositions are utilized on the skin of animals and humans to prevent the spread of disease.

One application in which such antimicrobial compositions are particularly useful is for the prevention and control of mastitis in dairy herds. Mastitis is a disease of the mammary glands typically caused by bacteria. Streptococcus agalactiae and Staphylococcus aureus probably account for the majority of all udder infections. These bacteria enter the teat through the teat orifice, migrate up through the teat canal, and produce inflammation of the milk-producing tissue. Other bacteria which are also of concern in mastitis infections include Streptococcus dysgalactiae, Aerobacter aerogenes, Pseudomonas aeruginosa, Salmonella enteritidis, Clostridium perfingens, Escherichia coli, and Corynebacteriium pyogenes. Mastitis infections can be spread from animal to animal in the dairy herd, and may alter the composition, quantity, appearance and quality of the milk. Animals which contract the disease are removed from service. Thus, this type of infection can be economically detrimental to a dairy operation.

Further, application of antimicrobial compositions reduces the risk of contamination of the milk by reducing the number of bacteria on the teat which can be transferred to the milk during the milking process.

It is thus clear to see that prevention and control of mastitis is extremely important to the economic health of a dairy operation, and that as a consequence, antimicrobial compositions are commonly used in dairy operations in udder washes, and in pre-milking and post-milking teat dips.

Teat dips are further classified as barrier and non-barrier teat dips. Barrier teat dips form a coating or film over the teat to provide a longer lasting barrier against mastitis-causing agents. Non-barrier teat dips typically have a more immediate, but shorter lasting efficacy.

A variety of antimicrobial products are currently available for topical treatment of mastitis including those based on chlorhexadine, iodine, chlorine (e.g. sodium hypochlorite) or a quaternary ammonium compounds. Iodine and chlorine based compositions are very commonly used. These compositions are usually administered to the teat after removal of the milking cup by dipping or spraying the teat. It is believed these bacteriocides kill a substantial number of bacteria including mastitis pathogens, and thus reduce the spread of bacteria into the mammary gland where mastitis may become evident. There are usually insignificant residual effects of these bacteriocides between milkings.

Carboxylic fatty acids are known as effective antimicrobials. For example, U.S. Pat. No. 4,002,775 to Kabara describes a food-grade microbicidal or microbiostatic composition containing a food or food-grade material and as the primary microbicide a monoester of a polyol and a twelve carbon atom aliphatic fatty acid.

U.S. Pat. No. 4,406,884 to Fawzi, et al. describes a topical antimicrobial composition in the form of an aqueous gel or lotion containing C₅-C₁₂ fatty acids and having a pH of no greater than about 5 and U.S. Pat. No. 4,343,798 to Fawzi, et al. describes topical antimicrobial anti-inflammatory compositions having a pH of less than about 5 and containing C₅-C₁₂ fatty acids together with a corticosteroid component.

U.S. Pat. No. 5,208,257 to Kabara describes a topical antimicrobial and anti-parasite pharmaceutical composition including a safe and effective amount of an ethoxylated or propoxylated glycerol fatty acid ester and a pharmaceutically acceptable carrier. Also disclosed is a topical antimicrobial pharmaceutical composition including a safe and effective amount of a tertiary mixture including a glycerol fatty acid ester, a binary mixture of fatty acids including a first fatty acid antimicrobial agent selected from C₆ to C₁₈ fatty acids, and a second fatty acid antimicrobial agent selected from C₆ to C₁₈ fatty acids, and a pharmaceutically acceptable carrier. Further disclosed is a topical antimicrobial pharmaceutical composition including a safe and effective amount of an ethoxylated or propoxylated glycerol fatty acid ester, a binary fatty acid mixture including a first fatty acid antimicrobial agent selected from C₆ to C₁₈ fatty acid and the second fatty acid antimicrobial agent selected form C₆ to C₁₈ fatty acids, where the second fatty acid is not the same as the first fatty acid, and a pharmaceutically acceptable carrier.

U.S. Pat. No. 4,404,040 to Wang describes sanitizing concentrate and use compositions including aliphatic short chain fatty acid, hydrotrope or solutbilizer for the fatty acid, hydrotrope-compatible acid so that the concentrate, when diluted with a major amount of water provides a use solution having a pH in the range of 2.0-5.0.

U.S. Pat. No. 5,330,769 to McKinzie, et al. describes fatty acid sanitizer concentrates and diluted final solutions which include individual amounts of a germicidally effective fatty acid, hydrotrope, strong acid sufficient to lower the pH of the final solutions to about 1-5, and a concentrate stabilizing weak acid component selected from the group consisting of propionic, butyric and valeric acids and mixtures thereof. Preferably, the fatty acid is a mixture of nonanoic and decanoic acids, whereas the strong acid is selected from the group consisting of orthophosphoric, sulfuric and mixtures thereof.

U.S. Pat. No. 5,308,868 to Kefford describe a teat dip composition in the form of an oil-in-water emulsion in which the oil phase includes an unsaturated fatty acid, for example linseed oil fatty acid, having a carbon chain length in the range of C₁₆ to C₂₀.

U.S. Pat. No. 5,462,714 to Talwalker, et al. describes a substantially non-corrosive antimicrobial composition that includes by weight percent between 0.25 to 2.0% available iodine, 20.0 to 50.0% fatty acid, 15.0%-35% non-ionic surfactant, 5.0-16.0% (w/v) buffering agent and 10.0-60.0% water (v/v), and methods of using such compositions. The composition has a pH between 3.0 and 5.0.

U.S. Pat. No. 5,569,461 to Andrews describes a topical composition and related method for disinfecting, cleansing, conditioning and treating skin using a propylene glycol monoester of capric or caprylic acid, a second propylene glycol monester of capric and/or caprylic acid, a synergist, propylene glycol, a surfactant and a vehicle.

However, antimicrobial materials vary widely in their efficacy and in the amount of skin irritation caused. Many of these materials are also viewed as corrosive to skin.

### SUMMARY OF THE INVENTION

The present invention provides improved topical antimicrobial compositions and methods for using such composition. The compositions are typically used without dilution, also known as “ready-to-use”.

The compositions include at least one at least one aliphatic short chain antimicrobially effective C₆ to C₁₀ fatty acid and a coupler. The coupler is suitably a sulfonate or sulfate coupler, and the fatty acid is suitably a C₇ fatty acid.

A viscosity modifying agent, as well as other additives, may be optionally employed.

The compositions of the present invention have been found to exhibit improved stability over those compositions which employ only the acid while maintaining excellent antimicrobial efficacy.

The antimicrobial compositions of the present invention are suitable for a broad range of applications, but have been found to be particularly useful for application to the teats and udders of dairy animals as udder and teat washes, and as pre-milking and post milking sanitizing solutions (pre-dips and post-dips). The compositions of the present invention have been found to produce less skin irritation.

### DETAILED DESCRIPTIONS OF THE PREFERRED EMBODIMENTS

While this invention may be embodied in many different forms, there are described in detail herein specific embodiments of the invention. This description is an exemplification of the principles of the invention and is not intended to limit the invention to the particular embodiments illustrated.

The term “short chain fatty acids” as used herein refer to those acids generally having from 6 to 14 carbon atoms, preferably from about 6 to 12 carbon atoms, more preferably about 6 to 10 carbon atoms and most preferably about 7 to 10 carbon atoms. In one specific embodiment of the present invention, the composition comprises at least one C₇ fatty acid.

The fatty acid is useful from about 0.25 wt-% to about 10 wt-%, and suitably from about 1 wt-% to about 5 wt-%.

In the present invention, a coupler or hydrotrope is employed in combination with the fatty acid. The coupler as employed in the present invention has been found to be particularly effective in solubilizing the fatty acid in an aqueous environment, while maintaining excellent antimicrobial efficacy. Thus, the compositions exhibit improved stability even at low viscosities. For compositions which employ only an acid, in contrast, the compatibility may be improved by raising the viscosity of the composition, but this is not always a desirable option. Furthermore, a majority of applications require lower viscosities.

In contrast to the couplers of the present invention, other hydrotropes and couplers, when employed in combination with the acid, have been found to actually reduce the antimicrobial efficacy of the composition. For example the employing phosphate esters, nonylphenols alcohol ethoxylates or alcohol ethoxylate carboxylates may dramatically diminish antimicrobial efficacy whereas the sulfonates of sulfates exhibit dramatically enhanced antimicrobial efficacy when used in combination with the fatty acids of the present invention.

A fatty acid by itself is not particularly soluble in water especially when other solutes such as skin conditioners are present and competing for the water as a solvent. A coupler, which may also be referred to as a hydrotrope, has the affect of solubilizing the fatty acid in an aqueous environment. Surprisingly, in the present invention, the coupler has been found to provide improved compatibility and stability in an aqueous composition, while maintaining excellent antimicrobial efficacy. The couplers are suitably sulfonates, sulfates, and the like.

One group of coupling agents which have been found to be particularly suitable in the present invention, providing effective antimicrobial kill when used in combination with the fatty acid of the present invention include a group of anionic surfactants which have a hydrophobic group of C₆₋₂₂ such as alkyl, alkylaryl sulfonates or sulfates.

More specifically, useful sulfonates, sulfates and corresponding disulfonates and sulfates include linear or branched C₆-C₁₄ alkylbenzene sulfonates, alkane sulfonates such as the alkali metal sulfonates and corresponding disulfonates of C₆-18 alkyl sulfonates including 1-octane sulfonate and 1,2-octane sulfonate, alkyl sulfates such as the sodium salts of octyl, dodecyl or lauryl sulfates, alkali metal aryl sulfonates, C₆₋₃₀ alkaryl sulfonates such as the linear alkyl benzene sulfonates, sodium C₂₋₁₈ alkyl naphthalene sulfonate and sodium xylene sulfonate, alkyl phenoxy benzene disulfonates, alkylaryl sulfonates, alkyl ether sulfates, alkylamidoether sulfates, alkylaryl polyether sulfates, monoglyceride sulfates, alkylamide sulfonates, olefinsulfonates, paraffin sulfonates, alkyl sulfosuccinates, alkyl ether sulfosuccinates, alkylamide sulfosuccinates, alkyl sulfosuccinamate, alkyl sulfoacetates. Generally, the alkyl or acyl radical in these various compounds comprise a carbon chain containing 12 to 20 carbon atoms, and so forth.

Such surfactants can be provided not only as alkali metal salts, but also, for instance, as ammonium salts, amine salts, amino alcohol salts, magnesium salts, and so forth.

Such compositions are known to the art, and are presently commercially available as anionic surface active agents, also referred to as anionic surfactants.

In some particular embodiments of the present invention, the couplers utilized include alkyl sulfonates such as 1-octane sulfonates available from Crompton Corp. under the tradename of WITCONATE® such as WITCONATE® NAS 1-octane sulfonate, NAXONATES® available from Ruetgers-Nease, and DOWFAXS® available from Dow Chemical Co.

In contrast, most classes of couplers have been found to have a “neutralizing” effect on the fatty acid which is undesirable herein. For instance, lecithin and polyoxyethylene (20) sorbitan monooleate such as TWEEN® 80 a nonionic ethoxylated sufactant/coupler available from Spectrum Chemical Manufacturing Corp. have such an effect. Additionally, NEODOL® 25-9 another alcohol ethoxylate; NEODOX® 25-6 an ethoxylate carboxylate surfactant/coupler, and NPE-12®, an alcohol ethoxylate of a nonylphenol available from Shell Chem. Co; PE 362® the phosphate ester of an alcohol ethoxylate available from Ecolab Inc. all have also been found to dramatically reduce the antimicrobial efficacy of the fatty acid, though they are very effective couplers. These effects appear to be consistent within classes of surfactant/hydrotropes.

Various optional ingredients known in the art may also be included in the compositions of the present invention including surfactants (both detersive including the soap type surfactants, and others) such as nonionic, zwitterionic, cationic and other anionic surfactants, defoamers, dyes or colorants, fragrance components or perfumes, other antimicrobials, antifungicides, viscosity control agents including thickeners or rheology modifiers, pH adjusters or buffers, emollients or other skin conditioners or softeners, humectants, anti-irritants, antioxidants, binders, chelating agents, film formers, plasticizers, preservatives, propellants, reducing agents, bleaches or brighteners, solvents, foam boosters, other hydrotropes, solubilizing agents, suspending agents (non surfactant), UV absorbers, Vitamin E, aloe, insect repellents, enzymes, other chemical additives, and so on and so forth. There are other optional ingredients more specific to certain types of formulations not mentioned herein such as those used in cosmetic formulations, detergents, fabric softeners and so on and so forth. These optional additives, as well as many others, are known to one of skill in the art and are selected based on the specific end use of the composition.

Suitable soap type surfactants include alkali metal, ammonium, alkanol ammonium salts of aliphatic alkane or alkene monocarboxylic acids having about 6 to about 20 carbon atoms, and preferably from about 8 to about 18 carbon atoms. The latter group includes alkanolammonium salts of lauric acid, myristic acid, palmitic acid, stearic acid, oleic acid, linoleic acid, ricinoleic acid, coconut fatty acid, palm kernel fatty acid and tallow fatty acid. Sodium, potassium ammonium, mono-, di-, and triethanolammonium cations or combinations thereof Soaps may be prepared by either direct saponification of fats and oils or by neutralization of free fatty acids.

Any of the nonionic surfactants of the conventional variety may be added in effective amounts, i.e., amounts which are shown to be effective. Exemplary nonionic surfactants include those having a hydrophobic moiety, such as C₈-C₂₀ primary or secondary, branched or straight chain monoalcohols, C₈-C₁₈ mono- or dialkyphenols, C6-C₂₀ fatty acid amides, and a hydrophilic moiety which consists of alkylene oxide units. These nonionic surfactants are for instance alkoxylation products of the above hydrophobic moieties, containing from 2 to 30 moles of alkylene oxide including ethylene, propylene, and butylene oxides, and mixtures thereof. Typical examples of such nonionic surfactants are C₉-C₁₁ primary, straight-chain alcohols condensed with 5-9 moles of ethylene oxide such as NEODOL® 25 (2.5-9 mole EO alcohol ethoxylates) available from Shell Chemical Co. in Houston, Tex., C₁₂-C₁₅ primary straight-chain alcohols condensed with from 6-12 moles of ethylene oxide, or with 7-9 moles of a mixture of ethylene oxide and propylene oxide, C₁₁-C₁₅ secondary alcohols condensed with from 3-15 moles of ethylene oxide, and C₁₀-C₁₈ fatty acid diethanolamides, and tertiary amine oxides such as higher alkyl di(lower alkyl or lower substituted alkyl)amine oxides.

More specifically, nonionic surfactants include the condensates of 2 to 30 moles of ethylene oxide with sorbitan mono- and tri-C₁₀-C₂₀ alkanoic acid esters having a HLB of 8, alcohol alkoxylates including the primary aliphatic alcohol ethoxylates, secondary aliphatic alcohol ethoxylates, alkylphenol ethoxylates such as nonylphenol ethoxylate and ethylene-oxide-propylene oxide condensates on primary alkanols, and condensates of ethylene oxide with sorbitan fatty acid esters.

Suitable amphoteric or zwitterionic surfactants include betaines and sulfobetaines including alkyl betaines (oleyl betaine and lauryl betaine), cocamidopropyldimethyl betaine, cocamido betaine, alkyl sultaines, alkyl amphoacetates (cocamphoacetate), alkyl amphodiacetates (cocamphodiacetate), alkyl amphopropionates, alkyl amphodipropionates (cocamphocarboxypropionate), cocamphocarboxy propionic acid, cocamidopropylhydroxysultaine, amine oxides, and so forth.

Suitable cationic surfactants include cetyl trimethyl ammonium chloride, trimethyl coco quaternary ammonium chloride, diquaternary polydimethylsiloxane, and trimethyl quaternary ammonium chloride. Exemplary suitable nonionic surfactants include alkanolamine, alkyldimethyl oxide, coconut monoethanolamine, cetyldimethylamine oxide, stearamine oxide, oleamine oxide, and cocamidopropylamine dimethyl oxide.

This list is not an exclusive list of surfactants and is only intended as exemplary and as a guide herein. Surfactants are discussed in detail in McCutcheon's Detergents and Emulsifiers, 1999, North American Edition, MC Publishing Co. incorporated by reference herein in its entirety. One of ordinary skill in the art would know how to select surfactants for use in such systems depending on the application, properties desired, as well as the other ingredients involved.

Teat dips and udder washes can result in irritation to the teat skin, and also teat cracking. This in turn can lead to further infections of the teat. Most aggressive, antimicrobial compositions are also very irritating to animal or human skin, surprisingly, the antimicrobial compositions of the present are quite nonirritating. However, emollients, skin conditioners and/or humectants may be added to the compositions. Humectants refer to those skin conditioners that help control the moisture content in the epidermis of the skin. Emollient can also be humectants. Generally, and water soluble or dispersible skin conditioning agent known to those of skill in the art may be used in the present invention. Exemplary conditioners include, but are not limited to, polyhydric alcohols including glycerin, glycols including polyethylene and polypropylene glycol homopolymers and copolymers, ethoxylated lanolins including LANETO® 50 polyethylene glycol (PEG) 75/lanolin, mannitol, sorbitol, fatty acid esters of simple monohydric alcohols including isopropyl palmitate or isopropyl myristate and similar esters, polyol esters of fatty acids, vegetable oils, and similar natural sourced derivatives such as Aloe, and so forth may be included in these compositions. Generally, the emollient is useful from about 0.5 to about 20 wt-% of the composition preferably about 1 to 10-wt % of the composition.

Humectants are another class of skin conditioners which help to control the moisture content in the epidermis of the skin. The emollients described above also function as a humectant.

Anti-irritants may also be optionally added to the compositions. Polyvinylpyrrolidone is an example of an anti-irritant.

Water soluble or dispersible dyes or colorants may be optionally added to the compositions of the present invention providing a composition having a sharp contrast to the skin, which permits the diary herd manager to visually discern that teats have been treated, for instance. Any dyes or colorants known to those of skill in the art may be utilized herein. Some examples include, but are not limited to FD&C Yellow #5, FD&C Blue #1 and FD&C Red #3.

Preservatives may be optionally added to the compositions of the present invention. Exemplary preservatives include sodium benzoate and benzyl alcohol. Preservatives are typically added to the compositions in small amounts of about 0.01 to 1.0 wt-%, more suitably about 0.05 wt-% to about 0.1 wt-% of the concentrate to retard bacterial growth and prolong usefulness of the compositions. Preservatives typically function by preventing deterioration of the protic acid component.

Thickeners, or viscosity or rheology modifiers may be added to the hard surface cleaning compositions according to the present invention in order to modify the viscous or thixatropic properties thereof. For example, in certain applications it is contemplated that it may be desirable to provide a more viscous, i.e. higher viscosities than that of water, either for aesthetic or for functional reasons. Suitable and commonly used thickeners include, but are not limited to, cellulosic compounds, xanthan gums, polymers, clays, and so forth.

Water soluble or water dispersible rheology modifiers useful herein may be either inorganic or organic. Inorganic thickeners are generally compounds such as colloidal magnesium aluminum silicate such as those trademarked as VEEGUM®, collodial clays (bentonites), or silicas such as those sold under the tradename of CAB-O-SIL® which have been fumed or precipitated to create particles with large surface to size ratios.

Useful organic thickeners include both natural and synthetic polymers including synthetic natural-based and synthetic petroleum-based. Synthetic petroleum-based water soluble polymers include, but are not limited to, polyvinylpyrrolidone, polyvinylmethylether, polyacrylic acid and polymethacrylic acid, polyacrylamide, polyethylene oxide, polyethyleneimine, and so forth.

Synthetic natural-based thickeners include cellulosic derivatives including, but not limited to, those wherein the free hydroxl groups on the linear anhydro-glucose polymers have been etherified or esterified to give a family of substances which dissolve in water and give viscous solutions such as alkyl and hydroxylalkylcelluloses such as methylcellulose, hydroxyethylmethylcellulose, hydroxypropylmethylcellulose, hydroxybutylmethylcellulose, hydroxyethylcellulose, ethylhydroxyethylcellulose, hydroxypropylcellulose, and carboxymethylcellulose.

Natural hydrogel thickeners of use include vegetable derived exudates such as tragacanth, karaya, and acacia gums and extractives such as caragheenan, locust bean gum, guar gum and pectin and pure culture fermentation products such as xanthan gum. Chemically, all of these materials are salts of complex acidic polysaccharides.

Any pH buffering compound or pH buffer composition which is compatible with the aqueous compositions taught herein may be used, and are well known to the art. Examples of useful buffers, include, but are not limited to, alkaline earth metal, ammonium and alkali metal carbonates, bicarbonates, silicates, polysilicates, metasilicates, phosphates such as ortho phosphates, polyphospates, pyrophosphates, triphosphates, tetraphosphates, carbonates, bicarbonates, hydroxides, and so forth, and mixtures thereof. It may also be suitable to use as buffers materials such as borates, aluminosilicates (zeolites), aluminates and certain organic materials such as gluconates, succinates, maleates, and their alkali metal salts, 2-amino-2-methyl-1-propanol (amino alcohols). This list is not exclusive and one of skill in the art would understand what compounds may be available for use as buffering agents.

The compositions of the present invention typically have a low pH of about 3-6. One advantage of the teat dip compositions of the present invention is that they do not require the addition of a strong acid, for example a mineral acid such as nitric, phosphoric or sulfuric. The fatty acid is itself sufficiently acidic to buffer the use pH to the efficacious pH range of <5. Alkaline agents such as acid salts, amines or hydroxides such as potassium hydroxide, may also be added to elevate the pH of the compositions of the present invention to above a pH at which the composition is no longer irritating. Such irritation typically occurs in the pH range below about 3.

Barrier type of teat dips further comprise a film former. Film formers useful herein include, but are not limited to, polyvinyl alcohols and polyvinylpyrrolidones.

This invention provides concentrated antimicrobial systems which may be diluted to use solutions for any sanitizing or cleaning composition, but in particular for udder washes and teat dips including both post- and pre-teat dips, and for barrier teat dips.

Further, it is possible to utilize the compositions not only as an udder wash, but also as a dip, e.g. predip, simply by varying the dilution rate of the concentrated antimicrobial system. Typically an udder wash is a mild sanitizing solution containing a small amount of germicide plus cleaning agents such as soaps or detergents whose principle purpose is to remove dirt, manure, and other debris that may be on the teats and udder prior to milking the cow. A predip, on the other hand, is a strong germidical solution which is used prior to milking to substantially reduce the population of pathogenic organisms on the teats that cause certain types of mastitis in dairy cows. The principle purpose of an udder wash is not to control or prevent disease in cattle, but to help ensure that no foreign material such as dirt, manure, bedding, etc., gets into the milk via the milking process.

Because of economic necessity, udder washes and teat dips are sold as concentrates to be diluted on the farm just prior to use. They are generally used at a use dilution of about 1 oz. (29.6 ml) concentrate to a gallon (3.785 liters) of water which is a ratio of about 1:128 to a use dilution of about 1 oz. (29.6 ml) of concentrate to four gallons (15.14 liters) of water which is a ratio of about 1:512. Large amounts of germicidal agents are required to be present in the concentrates so that an antimicrobially active solution results when the product is diluted for use.

The above disclosure is intended to be illustrative and not exhaustive. The description will suggest many variations and alternatives to those of ordinary skill in the art. All of these alternatives and variations are intended to be included within the scope of the attached claims. Those familiar with the art may recognize other equivalents to the specific embodiments described herein which equivalents are also intended to be encompassed by the claims attached hereto.

The following non-limiting examples further illustrate embodiments of the present invention.

TEST METHODS

Sanitizing Efficacy/Germicidal and Detergent Sanitizing Action of Disinfectants—Final Action

This test measures the sanitizing efficacy against Staphylococcus aureus ATCC 6538 and Escherichia coli ATCC 11229 with a 10% milk challenge at a 15 second exposure time. A 10% milk challenge is achieved by adding 90 ml of the test formulation to 10 ml of milk, mixing, and removing 1 ml for a final volume of 99 ml.

TABLE 1

| | Staphylococcus aureus ATCC 6538 |
|---|---|
| Test System | Escherichia coli ATCC 11229 |
| Organic soil | Sterile milk with 2% milk fat |
| Test temperature | ambient |
| Exposure time | 15 seconds |
| Neutralizer | 50:50 Chambers:1% sodium thiosulfate |
| Subculture medium | Tryptone glucose extract agar |
| Incubation | 37° C. for 48 hours |

### EXAMPLES

### Example 1

An antimicrobial composition of the present invention was prepared in both a pre-post and a barrier teat dip formulation. The fatty acid component and the coupler were kept at a constant level.

TABLE 2

| | | Pre-post | |
|---|---|---|---|
| | | teat | Barrier |
| Ingredient | Function | dip | teat dip |
| DI water | | 72.175 | 67.147 |
| Potassium hydroxide, 45% | buffer | 0.475 | 0.488 |
| Benzoic acid or salt | Preservative | 0.150 | 0.140 |
| KELZAN ® T | Thickener | 0.100 | 0.300 |
| xanthan gum | | | |
| PVP-NP-K30 | anti-irritant | 0.200 | 0.200 |
| polyvinylpyrrolidone | film former | | |
| NEODOL ® 25-9 mole EO | surfactant/coupler | 0.050 | 0.050 |
| alcohol ethoxylate | | | |
| RHODAFAC ® RE-610 | Coupler | 1.000 | 1.000 |
| WITCONATE ® | Coupler | 10.50 | 10.50 |
| NAS-FAL | | | |
| LANETO ® 50 | emollient/humectant | 0.100 | 0.100 |
| PEG-75 Lanolin | | | |
| Propylene glycol | emollient/humectant | 7.000 | 7.000 |
| Glycerine, 96% | emollient/humectant | 7.000 | 7.000 |
| Heptanoic acid, C₇ | antimicrobial | 1.000 | 1.000 |
| 10% ELVANOL ® 50-42 | film former | — | 5.000 |
| polyvinyl alcohol premix | | | |
| Lactic acid, 88% | exfoliating agent; skin | 0.250 | 0.075 |
| | conditioner, pH buffer | | |

The sanitizing efficacy of the formulations was then tested according to Test Method No. 1 described above.

Inocolum numbers were calculated three times and an average taken.

The average inoculum numbers for S. aureus were 7.1×10⁷ and the average inoculum numbers for E. coli were 8.3×10⁷.

TABLE 3
S. aureus

| | Survivors | Average Survivors | Log | Percent |
|---|---|---|---|---|
| | (CFU/mL) | (CFU/mL) | Reduction | Reduction |
| Pre-post | <10, <10 | <10 | >6.85 | >99.999 |
| Barrier | <10, <10 | <10 | >6.91 | >99.999 |

TABLE 4
E. coli

| | Survivors | Average Survivors | Log | Percent |
|---|---|---|---|---|
| | (CFU/mL) | (CFU/mL) | Reduction | Reduction |
| Pre-post | <10, <10 | <10 | >6.91 | >99.999 |
| Barrier | <10, <10 | <10 | >6.91 | >99.999 |

Both the pre-post and the barrier teat dips exhibited a >99.999 percent reduction against S. aureus (ATCC 6538) and E. coli (ATCC 11229) with a 10% milk challenge after 15 seconds exposure.

### Examples 2-10 and Comparative Examples A-F

To evaluate the effect of specific coupling agents upon the antimicrobial efficacy of the formulas a second set of examples were made using the base composition found in Table 5. Some of the examples were adjusted to a pH of 3 and some of the examples were adjusted to a pH of 4.5 using potassium hydroxide. The concentration of the coupler was varied at both pH levels.

TABLE 5

| | | Barrier-like teat dip base |
|---|---|---|
| Ingredient | Function | formula |
| DI water | | 67.147 |
| Potassium hydroxide, 45% | Buffer | ca 0.49 |
| Benzoic acid or salt | Preservative | 0.140 |
| KELZAN ® T xanthan | Thickener | 0.300 |
| gum | | |
| Heptanoic acid, C₇ | Antimicrobial | 0-0.3 |

TABLE 6
Examples 2-10 and Comparative Examples A-F

| | | | Heptanoic acid |
|---|---|---|---|
| Sample | pH | 1-octane sulfonate (w/w %) | (C₇, w/w %) |
| Comparative A | 4.5 | 0.0 | 0.0 |
| Comparative B | 4.5 | 0.0 | 0.3 |
| Example 2 | 4.5 | 0.1 | 0.3 |
| Example 3 | 4.5 | 0.2 | 0.3 |
| Example 4 | 4.5 | 0.4 | 0.3 |
| Example 5 | 4.5 | 0.8 | 0.3 |
| Example 6 | 4.5 | 1.0 | 0.3 |
| Example 7** | 4.5 | 2.0 | 0.3 |
| Comparative C | 4.5 | 2.0 | 0.0 |
| Example 8* | 4.5 | 2.0 | 0.3 |
| Comparative D** | 3.0 | 2.0 | 0.0 |
| Comparative E | 3.0 | 0.0 | 0.3 |
| Example 9 | 3.0 | 0.2 | 0.3 |
| Example 10** | 3.0 | 0.4 | 0.3 |

*A10 was augmented by the addition of 5% Nonylphenolethoxylate, 12 mole ethoxylate (NPE-12) to determine if it would negatively affect the antimicrobial activity of the 1-octane sulfonate and heptanoic acid mixture.
**Formulations used in neutralization controls.

Inocolum numbers were calculated three times and an average taken. The average inoculum numbers for S. aureus were 1.5×10⁸.

TABLE 7
Examples 2-10 and Comparative Examples A-F
Efficacy against S. aureus

| | | Average | | |
|---|---|---|---|---|
| | Survivors | Survivors | Log | Percent |
| | (CFU/mL) | (CFU/mL) | Reduction | Reduction |
| A | 115 × 10⁶, 124 × 10⁶ | 1.2 × 10⁸ | 0.10 | 20.000 |
| B | <1 × 10², <1 × 10² | <1 × 10² | >6.18 | >99.999 |
| Ex 2 | <1 × 10², <1 × 10² | <1 × 10² | >6.18 | >99.999 |
| Ex 3 | <1 × 10², <1 × 10² | <1 × 10² | >6.18 | >99999 |
| Ex 4 | <1 × 10², <1 × 10² | <1 × 10² | >6.18 | >99.999 |
| Ex 5 | <1 × 10², <1 × 10² | <1 × 10² | >6.18 | >99.999 |
| Ex 6 | <1 × 10², <1 × 10² | <1 × 10² | >6.18 | >99.999 |
| Ex 7 | <1 × 10², <1 × 10² | <1 × 10² | >6.18 | >99.999 |
| C | 118 × 10⁶, 89 × 10⁶ | 1.0 × 10⁸ | 0.18 | 33.333 |
| 8 | <1 × 10², <1 × 10² | <1 × 10² | >6.18 | >99.999 |
| D | 240 × 10⁴, 139 × 10⁴ | 1.9 × 10⁶ | 1.90 | 98.733 |
| E | <1 × 10², <1 × 10² | <1 × 10² | >6.18 | >99.999 |
| Ex 9 | <1 × 10², <1 × 10² | <1 × 10² | >6.18 | >99.999 |
| Ex 10 | <1 × 10², <1 × 10² | <1 × 10² | >6.18 | >99.999 |
| F | 87 × 10⁶, 96 × 10⁶ | 9.2 × 10⁷ | 0.21 | 38.667 |

While the heptanoic acid alone provides satisfactory antimicrobial efficacy, it exhibits less compatibility alone than when employed in combination with the 1-octane sulfonate coupler. The compatibility and thus stability may be increased by increasing the viscosity of the composition, but higher viscosity is not desirable for many applications.

### Examples 11-16 and Comparative Examples G-N

The barrier type of teat dip composition found in the following Table 8 was used to prepare a series of examples and comparative examples employing various couplers. The compositions were buffered to a pH of 3 or 4.5 with potassium hydroxide. The compositions were then tested for antimicrobial efficacy and the results are shown in Table 11.

TABLE 8

| | | Barrier-type |
|---|---|---|
| | | teat dip base formula |
| Ingredient | Function | (%) |
| DI water | | 88-99 |
| Potassium hydroxide, 45% | Buffer | ca 0.49 |
| Benzoic acid or salt | Preservative | 0.140 |
| KELZAN ® T | Thickener | 0.300 |
| xanthan gum | | |
| Heptanoic acid, C₇ | Antimicrobial | 0-0.3 |
| COUPLER | COUPLING | 0-10 |

TABLE 9

| | Products | |
|---|---|---|
| Generic | Name | Chemical name |
| sodium octane sulfonate | NAS-FAL | 1-octane sulfonate, sodium salt |
| (NAS) | | |
| alcohol ethoxylate | NEODOX ® | C₁₂-C₁₅ linear primary alcohol |
| carboxylate | 25-6 | ethoxylate (2EO) carboxylate |
| (AEC) | | |
| sodium xylene sulfonate | STEPANATE ® | Sodium xylene sulfonate |
| (SXS) | SXS | |
| phosphate ester | PS 236 | C₁₂-C₁₅ linear primary alcohol |
| (PE) | | phosphate ester |
| alcohol ethoxylate | NEODOL ® 25-9 | C₁₂-C₁₅ linear primary alcohol |
| (AE) | | 9 mole ethoxylate |
| nonyl phenol ethoxylate | SURFONIC ® | nonyl phenol ethoxylate |
| (15 moles ethylene oxide) | L24-15 | (15 moles |
| (NPE-15) | | ethylene oxide) |

NEODOX ® 25-6 and NEODOL ® 25-9 are available from Shell Chemical Co.
SURFONIC ® L24-15 is available from Huntsman Chemical Co.
STEPANATE ® SXS is available from Stepan Co.
NAS-FAL is available under the tradename of WITCONATE ® from Crompton Corp. and also available as pure 1-octane sulfonic acid, sodium salt, from Aldrich Chemical Co.
PS-236 is available under the tradename of EMPHOS ® from Crompton Corp.

TABLE 10

| | | | Heptanaic acid |
|---|---|---|---|
| Sample | pH | COUPLER (w/w %) | (C₇, w/w %) |
| Comp Example G | 4.5 | 0.0% NAS | 0.3 |
| Example 11 | 4.5 | 0.8% NAS | 0.3 |
| Example 12 | 4.5 | 2.4% NAS | 0.3 |
| Example 13 | 4.5 | 8% NAS | 0.3 |
| Example 14 | 3.0 | 10% NAS | 0.0 |
| Example 15 | 3.0 | 10% NAS | 0.3 |
| Comp Example H | 3.0 | 0% NAS | 0.3 |
| Comp Example I | 4.5 | 5% AEC | 0.0 |
| Comp Example 16 | 4.5 | 6% SXS | 0.3 |
| Comp Example J | 4.5 | 5% PE | 0.3 |
| Comp Example K | 4.5 | 5% AE | 0.3 |
| Comp Example L | 4.5 | 5% AEC | 0.3 |
| Comp Example M | 4.5 | 5% NPE-15 | 0.3 |
| Comp Example N | 4.5 | 0% coupler | 0.0 |

Inocolum numbers were calculated three times and an average taken. The average inoculum numbers for S. aureus were 1.2×10⁶.

The antimicrobial efficacy of each of the above compositions was tested and the results are found in the following Table 9.

TABLE 11
Antimicrobial Efficacy
S. Aureus

| | Survivors | Average Survivors | Log | Percent |
|---|---|---|---|---|
| | (CFU/mL) | (CFU/mL) | Reduction | Reduction |
| G | 132, 1002 × 10² | 5.7 × 10⁴ | 3.32 | 99.952 |
| 11 | <1, <1 × 10² | <1 × 10² | >6.08 | >99.999 |
| 12 | <1, <1 × 10² | <1 × 10² | >6.08 | >99.999 |
| 13 | <1, <1 × 10² | <1 × 10² | >6.08 | >99.999 |
| 14 | 3, <1 × 10² | <2 × 10² | >5.78 | >99.999 |
| 15 | <1, <1 × 10² | <1 × 10² | >6.08 | >99.999 |
| H | <1, <1 × 10² | <1 × 10² | >6.08 | >99.999 |
| I | 82, 54 × 10⁶ | 6.8 × 10⁷ | 0.25 | 43.333 |
| 16 | <1, <1 × 10² | <1 × 10² | >6.08 | >99.999 |
| J | 49, 54 × 10⁶ | 5.2 × 10⁷ | 0.36 | 56.667 |
| K | 81, 80 × 10⁶ | 8.0 × 10⁷ | 0.18 | 33.333 |
| L | 63, 56 × 10⁶ | 6.0 × 10⁷ | 0.30 | 50.000 |
| M | 101, 92 × 10⁶ | 9.6 × 10⁷ | 0.10 | 20.000 |
| N | 83, 97 × 10⁶ | 9.0 × 10⁷ | 0.12 | 25.000 |

Examples 11-16, the compositions which employed a combination of heptanoic acid and a sulfonate coupler exhibited excellent antimicrobial activity. Comparative example H which has heptanoic acid only, with no coupler, also exhibited good antimicrobial activity. Again, while the antimicrobial efficacy is good, compatibility is improved in those compositions which employ the sulfonate coupler according to the present invention in combination with heptanoic acid.

Some couplers when employed in combination with heptanoic acid, actually resulted in a composition having decreased antimicrobial activity over those compositions having heptanoic acid only. See Comparative Examples J-M which employed PE, AE, AEC, and NPE-15 couplers.

1. A ready-to-use teat dip composition comprising an antimicrobially efficacious combination of:
a) at least one fatty acid; and
b) at least one sulfonate or sulfate hydrotrope-solubilizer, or mixture thereof;
wherein said teat dip composition is free of a mineral acid.

2. The composition of claim 1 further comprising at least one viscosity modifying agent.

3. The composition of claim 1 wherein said viscosity modifying agent can raise viscosity to the range of 50-2000 cps.

4. The composition of claim 1 wherein said at least one fatty acid is an aliphatic short chain fatty acid.

5. The composition of claim 1 wherein said at least one fatty acid is a C₆ to C₁₀ fatty acid.

6. The composition of claim 1 wherein said at least one fatty acid is a C₇ fatty acid.

7. The composition of claim 1 wherein said at least one coupling agent is an aryl, alkyl or alkylaryl sulfonate or mixture thereof.

8. The composition of claim 1 wherein said coupling agent is a mixture or an alkyl and an alkylaryl sulfonate.

9. The composition of claim 1 wherein said at least one coupling agent is a C₃ to C₂₀ alkyl sulfonate, a C₃ to C₂₀ alkyl aryl sulfonate, or mixture thereof.

10. The composition of claim 9 wherein said alkyl aryl sulfonate is a xylene sulfonate, a cumene sulfonate, a toluene sulfonate, a napthyl sulfonate or a mixture thereof.

11. The composition of claim 9 wherein said at least one coupling agent is 1-octane sulfonate.

12. The composition of claim 1 comprising from about 0.25 wt-% to about 10 wt-% of said at least one fatty acid.

13. The composition of claim 1 comprising from about 5 wt-% to about 15 wt-% of said at least one sulfonate or sulfate hydrotrope-solubilizer or mixture thereof.

14. The composition of claim 1 wherein said composition has a pH of about 2.5 to about 6.

15. The composition of claim 1 further comprising water.

16. The composition of claim 1 wherein said teat dip composition is a barrier teat dip or a pre-post teat dip.

17. The composition of claim 1 wherein said composition exhibits antimicrobial efficacy against Staphylococcus aureus and Escherichia coli.

18. A method for treating or preventing bovine mastitis which includes the step of applying the composition of claim 1 to the teats of a cow.

19. The composition of claim 18 wherein said composition exhibits an antimicrobial efficacy of >6 log kill with a 15 sec exposure time at ambient temperature and a 10% milk challenge.

20. An antimicrobial composition comprising:
a) at least one aliphatic antimicrobially effective C₇ fatty acid; and
b) at least one coupling agent;
wherein said antimicrobial composition is free of a mineral acid.

21. The composition of claim 20 wherein said at least one coupling agent is a sulfonate, sulfate or mixture thereof.

22. The composition of claim 21 wherein said at least one coupling agent is an alkyl, aryl or alkyl aryl sulfonate or mixture thereof.

23. The composition of claim 22 wherein said at least one coupling agent is 1-octane sulfonate.

24. The composition of claim 20 said composition having a pH of greater than about 3.

25. The composition of claim 20 said composition having a pH of about 4 to about 4.5.

26. The composition of claim 20 comprising from about 0.25 wt-% to about 10 wt-% said fatty acid.

27. The composition of claim 20 comprising from about 5 wt-% to about 15 wt-% of said coupling agent.

28. The composition of claim 20 further comprising water.

29. The composition of claim 20 further comprising at least one selected from emollients, film-formers, thickeners, surfactants, chelating agents, dyes, perfumes, excipients, preservatives, antioxidants, alcohols, parabens, phenolics, astringents, antifungals, stabilizers, buffering agents, and mixtures thereof.

30. The composition of claim 20 wherein said antimicrobial composition is a sanitizer, cleanser or lotion.

31. An antimicrobial composition consisting essentially of:
a) at least one aliphatic short chain antimicrobially effective C₇ fatty acid; and
b) at least one coupling agent which is a sulfate or sulfonate.

32. The composition of claim 31 wherein said at least one coupling agent is 1-octane sulfonate.

33. A method of using a composition as a teat dip comprising applying to a mammalian teat a composition comprising at least one aliphatic short chain antimicrobial effective fatty acid and at least one sulfate or sulfonate hydrotrope-solubilizer.
